SEQUENCE LISTING (11 sequences)

SEQ ID NO: 1 - protein, 123 aa, Artificial Sequence
  Modified Cys
  MUTAGEN (6)..(6): Amino acid other than Cys, preferably Ala
  MUTAGEN (28)..(28): Amino acid other than Cys, preferably Ala
  MUTAGEN (61)..(61): Amino acid other than Cys, preferably Ala
  MUTAGEN (73)..(73): Amino acid other than Cys, preferably Ala
  MUTAGEN (77)..(77): Amino acid other than Cys, preferably Ala
  MUTAGEN (91)..(91): Amino acid other than Cys, preferably Ala
  MUTAGEN (111)..(111): Amino acid other than Cys, preferably Ala
  MUTAGEN (120)..(120): Amino acid other than Cys, preferably Ala
KQFTKXELSQLLKDIDGYGGIALPELIXTMFHTSGYDTQAIVENNESTEYGLFQISNKLWXKSSQVPQSRNIXDISXDKFLDDDITDDIMXAKKILDIKGIDYWLAHKALXTEKLEQWLXEKL

SEQ ID NO: 2 - protein, 123 aa, Artificial Sequence
  Modified Cys
KQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 3 - protein, 124 aa, Artificial Sequence
  Modified Cys
  MUTAGEN (7)..(7): the amino acid is an amino acid other than Cys, preferably Ala
  MUTAGEN (29)..(29): the amino acid is an amino acid other than Cys, preferably Ala
  MUTAGEN (62)..(62): the amino acid is an amino acid other than Cys, preferably Ala
  MUTAGEN (74)..(74): the amino acid is an amino acid other than Cys, preferably Ala
  MUTAGEN (78)..(78): the amino acid is an amino acid other than Cys, preferably Ala
  MUTAGEN (92)..(92): the amino acid is an amino acid other than Cys, preferably Ala
  MUTAGEN (112)..(112): the amino acid is an amino acid other than Cys, preferably Ala
  MUTAGEN (121)..(121): the amino acid is an amino acid other than Cys, preferably Ala
MKQFTKXELSQLLKDIDGYGGIALPELIXTMFHTSGYDTQAIVENNESTEYGLFQISNKLWXKSSQVPQSRNIXDISXDKFLDDDITDDIMXAKKILDIKGIDYWLAHKALXTEKLEQWLXEKL

SEQ ID NO: 4 - protein, 124 aa, Artificial Sequence
  Modified Cys
MKQFTKAELSQLLKDIDGYGGIALPELIATMFHTSGYDTQAIVENNESTEYGLFQISNKLWAKSSQVPQSRNIADISADKFLDDDITDDIMAAKKILDIKGIDYWLAHKALATEKLEQWLAEKL

SEQ ID NO: 5 - protein, 39 aa, Artificial Sequence
  Modified Cys
  MUTAGEN (6)..(6): the amino acid is an amino acid other than Cys, preferably Ala
  MUTAGEN (28)..(28): the amino acid is an amino acid other than Cys, preferably Ala
KQFTKXELSQLLKDIDGYGGIALPELIXTMFHTSGYDTQ

SEQ ID NO: 6 - protein, 39 aa, Artificial Sequence
  Modified Cys
KQFTKAELSQLLKDIDGYGGIALPELIATMFHTSGYDTQ

SEQ ID NO: 7 - protein, 43 aa, Artificial Sequence
  Modified Cys
  MUTAGEN (11)..(11): the amino acid is an amino acid other than Cys, preferably Ala
  MUTAGEN (31)..(31): the amino acid is an amino acid other than Cys, preferably Ala
  MUTAGEN (40)..(40): the amino acid is an amino acid other than Cys, preferably Ala
LDDDITDDIMXAKKILDIKGIDYWLAHKALXTEKLEQWLXEKL

SEQ ID NO: 8 - protein, 43 aa, Artificial Sequence
  Modified Cys
LDDDITDDIMAAKKILDIKGIDYWLAHKALATEKLEQWLAEKL

SEQ ID NO: 9 - protein, 23 aa, Artificial Sequence
  Derived from COPII family protein SAR1
MAGWDIFGWFRDVLASLGLWNKH

SEQ ID NO: 10 - protein, 15 aa, Artificial Sequence
  Fragment
KQFTKAELSQLLKDI

SEQ ID NO: 11 - protein, 15 aa, Artificial Sequence
  Fragment
MAGWDIFGWFRDVLA

(19) United States
(12) Patent Application Publication
(10) Pub. No.: US 2023/0372450 A1
(43) Pub. Date: Nov. 23, 2023

(54) METHOD FOR PREPARING A COMPOSITION COMPRISING AN UNFOLDED PROTEIN

(71) Applicant: LINNANE PHARMA AB, Malmö (SE)

(72) Inventors: Catharina SVANBORG, Malmö (SE); Tran Thi HIEN, Lund (SE)

(73) Assignee: LINNANE PHARMA AB, Malmö (SE)

(21) Appl. No.: 18/030,352

(22) PCT Filed: Oct. 5, 2021
(86) PCT No.: PCT/EP2021/077409

§ 371 Date: Apr. 5, 2023

(30) Foreign Application Priority Data
Oct. 6, 2020 (GB) 2015836.6

(51) Int. Cl. A61K 38/38; A61K 47/12; A61K 38/46; A61P 35/00

(52) U.S. Cl. CPC A61K 38/38; A61K 47/12; A61K 38/465; A61P 35/00; C12Y 301/01048

(57) ABSTRACT

The invention provides a substantially water-free composition for use in preparing a biologically active complex, methods for preparing such compositions and to uses thereof.

### TECHNICAL FIELD

The present invention relates to methods for preparing compositions that can in turn be used for rapid and simple preparation of biologically active complexes that have therapeutic activity, in particular in the treatment of tumours or as antibacterial or antiviral agents. The present invention further relates to methods of treating tumors and cancers, in particular to methods for selectively targeting tumor cells in preference to healthy cells, as well as to complexes and compositions for use in these methods.

### BACKGROUND

There has been much interest in the production of complexes that involve partially unfolded proteins and lipids. These proteins may have drastically different properties and particularly biological properties than the corresponding proteins in a fully folded state. The gain of new, beneficial function upon partial protein unfolding and fatty acid binding is a remarkable phenomenon, and may reflect a significant generic route of functional diversification of proteins via varying their conformational states and associated ligands. Thus, in addition to alternative splicing of mRNA transcripts, post-translational modifications and changes in tertiary structure of specific domains, partial unfolding of a previously native protein is becoming recognized as a mechanism to generate functional diversity. This may be due to a cellular response to unfolded proteins and to the lipid cofactor, which defines their altered properties. However, this response may be different in for instance tumour cells, which means that they may give rise to therapeutic potential. In order to form stable moieties, the unfolded proteins are frequently modified in some way, and in particular may be bound to cofactors such as fatty acid cofactors. The complexes formed in this way may be stable and give rise to therapeutic options.

HAMLET (human alpha-lactalbumin made lethal to tumor cells) is one such example of a new family of tumoricidal molecules, with remarkable properties. Formed from partially unfolded α-lactalbumin and with oleic acid as an integral constituent, HAMLET was discovered by serendipity when studying the ability of human milk to prevent bacteria from binding to cells. Early in vitro experiments showed that HAMLET displays broad anti-tumor activity with a high degree of tumor selectivity and subsequent therapeutic studies have confirmed HAMLET's tumoricidal activity and relative selectivity for tumor tissue in vivo. In a placebo controlled clinical study, topical HAMLET administration removed or reduced the size of skin papillomas and in patients with bladder cancer, local instillations of HAMLET caused rapid death of tumor cells but not of healthy tissue surrounding the tumor. Therapeutic efficacy of HAMLET in bladder cancer was recently demonstrated in a murine bladder cancer model and HAMLET treatment delayed tumor progression and led to increased survival in a rat glioblastoma xenograft model without evidence of cell death in healthy brain tissue. HAMLET thus appears to identify death pathways that are conserved in tumor cells, thereby distinguishing them from healthy, differentiated cells. More recently, a complex of the Alpha1H fragment of HAMLET and oleate showed promising results in Phase I/II clinical trials, revealing significant differences between treatment and placebo groups for several efficacy variables. Treatment was also shown to be safe with no drug-related side effects observed.

Other complexes using equine lysozyme and oleic acid have also been found to produce cell death (Vukojevic et al. Langmuir, 2010, 26(18) 14782-14787), suggesting that different, unfolded proteins can become cytotoxic when coupled to a suitable cofactor. Further studies have involved biologically active complexes formed from oleate complexed with proteins including coat complexes such as COPI, COPII (such as SAR 1), HOPS/CORVET, SEA (Seh1-associated), and clathrin complexes BAR domain proteins such as endophilins, and the ESCRT complex, including Snf7 domain subunits. Biologically active complexes formed from SAR1 and oleate have been of particular interest (see, for example, WO 2018/116165).

Other work focuses on the use of peptide fragments of these proteins which may also be used (see for example EP-B-2643010, WO 2018/210759, WO 2018/116165 and WO 2019/066409).

Classically, these types of complex were prepared as described by Svensson et. al, (2000). Proc Natl Acad Sci U.S.A. 97, 4221-4226. Native α-lactalbumin was purified from human milk by hydrophobic interaction chromatography. The protein was unfolded with EDTA, subjected to ion-exchange chromatography on a matrix pre-conditioned with oleic acid and eluted with high salt, specifically 1M NaCl to obtain biologically active complexes. Procedures of this type have been used to produce other biologically active complexes including BAMLET, from bovine alpha-lactalbumin, and complexes formed from recombinant forms of alpha-lactalbumin, in particular those without cysteine residues as described in WO 2010/079362.

An alternative preparation for such biologically active complexes is described in WO2010/131010. In this reference, BAMLET is prepared in a one-phase system, in which α-lactalbumin is reconstituted in phosphate buffered saline (PBS) and sodium oleate added. The mixture is then heated to temperatures at or above 60° C. and active complex obtained. This method has the advantage of being simple to carry out, and may even be carried out in-situ in a clinical situation with the assistance of kits.

Investigations have been conducted into the impact of the mix of buffer salts used in the preparation of the compound and it was found that the precise nature of the salts used in the production can impact on the activity of the product (see, for example, WO 2018/210759).

These previously described methods provide the biologically active complexes in aqueous solution, and an aqueous solution is not ideal for storage and transportation. As such, in other references, biologically active complex is prepared in solution and then lyophilized to a dry composition comprising the biologically active complex. The aqueous preparation is then reconstituted by dissolution of the lyophilized complex in, for instance, PBS (see for example WO2010/079362) before use.

The present invention provides an improved method of preparing a composition which is provides for simple manufacturing processes and is well suited for storage and transportation.

### SUMMARY OF THE INVENTION

According to the present invention there is provided method for preparing a composition, the method comprising the steps of combining: a) a dried polypeptide, b) fatty acid or lipid, or a pharmaceutically acceptable salt thereof, and c) a buffer component comprising at least two salts, preferably wherein the first salt is sodium or potassium chloride and the second salt is disodium phosphate or mono-potassium phosphate, wherein the composition is substantially water-free

The present invention also provides a composition obtainable by this method. The present invention provides a composition that is substantially water-free comprising: a) a dried polypeptide, b) fatty acid or a lipid, or a pharmaceutically acceptable salt thereof, and c) a buffer component comprising at least two salts, preferably wherein the first of which is sodium or potassium chloride and the second of which is disodium phosphate or mono-potassium phosphate, wherein the composition is substantially free of polypeptide that is in a complex with the oleic acid or a pharmaceutically acceptable salt thereof.

Also provided is a method for preparing a biologically active complex from the compositions of the invention.

The present invention arises from the surprising finding that, as long as the buffer salts are in accordance with the invention, the simple addition of water is all that is required for the polypeptide to undergo the precise level of unfolding required to form the biologically active complex with the fatty acid or lipid component. It is also noted that the polypeptide is observed to survive direct contact with solid buffer component salts and the high concentrations and concentration gradients that occur during the initial states of dissolution in water. In studies conducted by the applicant, comparable biological activity was achieved using the composition of the invention as compared with prior art techniques.

A significant advantage over the prior art is that the dry composition is made without the need for lyophilization. Lyophilization is an energy intensive process and requires multiple and time-consuming steps. By circumventing the need for lyophilization, the method of the invention is simpler and more economic, which greatly enhances the manufacturing process. In comparison with the lyophilization technique, it is particularly surprising that the biological activity is not compromised by using the dry compositions of the invention. Lyophilized compositions contain the biologically active complex in a dry state, meaning that the biologically active complex remains as such when reconstituted in water. In contrast, the compositions of the invention are substantially free from biologically active complex. By including the buffer component, the formulation provides the surprising ability to generate biologically active complex in situ, at a comparable level of activity to previous techniques.

Another significant advantage over the prior art is that the buffer component can be optimized and standardized at the point of manufacture. This means that in the simplest case the only requirement of the end user in order to successfully form the desired biologically active complex is to obtain and add water in an appropriate quantity, and agitate. While water is the simplest aqueous carrier, it will be appreciated that other aqueous carriers such as aqueous solutions and suspensions, or even gels, can be used with the composition of the invention to generate biologically active complex.

Where the biologically active complex is for pharmaceutical use, sterile water will typically be used. Significantly, the biologically active complex can be prepared by simple hand shaking and at ambient temperature, meaning preparation can be done conveniently at home or at the bedside without the need for specialist equipment.

Where the biologically active complex is for nutraceutical use, non-sterile water can be used and it is also possible that other aqueous solutions or suspensions, such as drinks, juices or dairy products, can be used to form the biologically active complex. Furthermore, because the compositions are water-free, they can readily be combined with other dry powder nutraceutical formulations (such as protein shakes).

The buffer component comprises at least two salts. Where the oleic acid is provided in the form of a pharmaceutically acceptable salt, the buffer component salts are in addition to the oleate salt. The salts of the buffer component can be combined into the composition as a single buffer component, or each salt can be combined individually into the composition.

By ‘dried polypeptide’ we are referring to a polypeptide that has been dried from a liquid phase, i.e. typically the liquid medium where it was synthesized, expressed, or purified. Typically, the dried polypeptide will be a polypeptide in powder form. The dried polypeptide will be substantially free from polypeptide that is in a complex with the fatty acid or lipid, or pharmaceutically acceptable salt thereof.

By ‘water-free’ we are referring to a composition that is substantially anhydrous, i.e. where there is an insufficient number or concentration of water molecules to form bulk water (such as liquid water, i.e. a water droplet). In other words, the composition is not in the aqueous phase. Water molecules can therefore be present in the composition (such as in the form of waters of crystallization within the polypeptide, or as water impurities within a reagent) as long as there is not sufficient water to form bulk water. The ‘water-free’ composition is also referred to herein as a ‘dry’ composition. Typically, the water content of the composition will be below about 1%, 0.1%, 0.01% or 0.001% w/w of water with respect to the other components.

In a particular embodiment, the buffer component further comprises a third salt which is mono-sodium or mono-potassium phosphate, and in particular is mono-potassium phosphate.

This composition is therefore easy to prepare in a variety of manufacturing and non-manufacturing environments.

The term “polypeptide” used herein includes proteins and peptides including long peptides.

Suitable polypeptides include naturally-occurring proteins, in particular alpha-lactalbumin, SAR1, lysozyme or other proteins having a membrane perturbing activity, recombinant proteins and in particular variants of said naturally-occurring proteins which lack intra-molecular bonds for example as a result of mutation of cysteine residues, or in particular, fragments of any of these proteins, in particular peptides of up to 50 amino acids.

The expression “active variant” refers to proteins or polypeptides having a similar biological function but in which the amino acid sequence differs from the base sequence from which it is derived in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

| Class | Amino acid examples |
|---|---|
| Nonpolar: | A, V, L, I, P, M, F, W |
| Uncharged polar: | G, S, T, C, Y, N, Q |
| Acidic: | D, E |
| Basic: | K, R, H. |

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions are possible provided that these do not interrupt the function of the DNA binding domain polypeptides.

Broadly speaking, fewer non-conservative substitutions will be possible without altering the biological activity of the polypeptides.

Determination of the effect of any substitution (and, indeed, of any amino acid deletion or insertion) is wholly within the routine capabilities of the skilled person, who can readily determine whether a variant polypeptide retains the fundamental properties and activity of the basic protein. For example, when determining whether a variant of the polypeptide falls within the scope of the invention, the skilled person will determine whether complexes comprising the variant retain biological activity (e.g tumour cell death) of complexes formed with unfolded forms of the native protein and the polypeptide has at least 60%, preferably at least 70%, more preferably at least 80%, yet more preferably 90%, 95%, 96%, 97%, 98%, 99% or 100% of the native protein.

Variants of the polypeptide may comprise or consist essentially of an amino acid sequence with at least 70% identity, for example at least 75%, 80%, 85%, 90%, 91%, 92%, 93%, 94%, 96%, 97%, 98% or 99% identity to a native protein sequence such as an alphalactalbumin or lysozyme sequence.

The level of sequence identity is suitably determined using the BLASTP computer program with the native protein sequences as the base sequence. This means that native protein sequences form the sequence against which the percentage identity is determined. The BLAST software is publicly available at http://blast.ncbi.nlm.nih.gov/Blast.cgi (accessible on 10 May 2017).

In one embodiment, the polypeptide or variant is a peptide that has no more than 200, 180, 160, 140, 120 or 100 amino acids. In a particular embodiment, the polypeptide or variant or fragment is a peptide that has no more than 50 amino acids, preferably no more than 45 amino acids. In another or the same embodiment, the polypeptide or variant or fragment is a peptide having at least 5, more preferably at least 10 amino acids. For example, the peptide may have from 10-45 amino acids. Such complexes are easier to prepare and the starting materials are less costly. For instance, peptides may be prepared using conventional methods for the production of peptides. The complexes formed may be easier to handle and formulate for administration, due to the smaller molecular weight.

It is suitably derived from a naturally occurring protein or a variant thereof. Suitable proteins are those identified as being active in such complexes, such as alpha-lactalbumin, SAR1, beta-lactoglobulin or lysozyme, but may be derived from any membrane perturbing proteins.

Membrane perturbing proteins are proteins which have the capability of interacting with the interface of cell membranes, in particular causing disruption such as tubulation of the cell membrane. Typically, the protein will become embedded in the cell membrane.

As set out in WO 2019/243547, polypeptides that are suitable for formation of biologically active complexes according to the invention can be defined by the following. When present in the complex, the polypeptide has an increased conformational fluidity of three-dimensional structure as compared to the polypeptide alone. This can be indicated by an increased peak width on at least some ¹H NMR peaks of the complex as compared to the corresponding width of the peaks of a ¹H NMR of the peptide alone. This increase in conformational fluidity can be further indicated by at least one of:
a. an increased transverse relaxation rate (R₂), as obtained by NMR as described herein, as compared to the corresponding transverse relaxation rate of the peptide alone;
b. a hydrodynamic radius 1.5 to 2.5 times larger than the corresponding hydrodynamic radius of the peptide alone; or
c. mean residue ellipticity (MRE), [θ], in deg cm² dmol⁻¹ as obtained using circular dichroism (CD) as described herein, at 220 nm that is at least 1 deg cm² dmol⁻¹ lower than the peptide alone.

In particular, the polypeptide can be derived from the alpha-helical domain of a naturally occurring protein as described above. The alpha-helical domain of said proteins would be well understood in the art or may be determined using conventional methods.

Where the polypeptide or alpha-helical domain contains cysteine residues, these may, in some embodiments, be modified to a different amino acid residue, such as an alanine residue, in order to avoid inter-molecular disulphide bonds.

The peptide suitably contains no elements that give rise to folding and therefore suitably lacks amino acids that give rise to intramolecular bonding such as cysteine residues. In particular, where the peptide is derived from a naturally occurring protein, any cysteine residues are replaced by other amino acids such as alanine.

In a particular embodiment, the polypeptide comprises or consists of an amino acid sequence according to any of SEQ ID Nos 1 to 11. Where an amino acid is represented by X below, the amino acid is an amino acid other than Cys, preferably Ala, to prevent intra-molecular bonding.

| SEQ ID | | |
|---|---|---|
| NO | Brief Description | Sequence |
| 1 | Human alpha- | KQFTKXELSQ LLKDIDGYGG IALPELIXTM FHTSGYDTQA |
| | lactalbumin - | IVENNESTEY GLFQISNKLW XKSSQVPQSR NIXDISXDKF |
| | modified Cys | LDDDITDDIM XAKKILDIKG IDYWLAHKAL |
| | | XTEKLEQWLX EKL |
| 2 | Human alpha- | KQFTKAELSQ LLKDIDGYGG IALPELIATM FHTSGYDTQA |
| | lactalbumin - Cys to | IVENNESTEY GLFQISNKLW AKSSQVPQSR |
| | Ala | NIADISADKF LDDDITDDIM AAKKILDIKG IDYWLAHKAL |
| | | ATEKLEQWLA EKL |
| 3 | Human alpha- | MKQFTKXELSQ LLKDIDGYGG IALPELIXTM |
| | lactalbumin - leading | FHTSGYDTQA IVENNESTEY GLFQISNKLW |
| | M - modified Cys | XKSSQVPQSR NIXDISXDKF LDDDITDDIM XAKKILDIKG |
| | | IDYWLAHKAL XTEKLEQWLX EKL |
| 4 | Human alpha- | MKQFTKAELSQ LLKDIDGYGG IALPELIATM |
| | lactalbumin - leading | FHTSGYDTQA IVENNESTEY GLFQISNKLW |
| | M - Cys to Ala | AKSSQVPQSR NIADISADKF LDDDITDDIM |
| | | AAKKILDIKG IDYWLAHKAL ATEKLEQWLA EKL |
| 5 | Alpha-lactalbumin | KQFTKXELSQ LLKDIDGYGG IALPELIXTM FHTSGYDTQ |
| | Alpha1 39mer - | |
| | modified Cys | |
| 6 | Alpha-lactalbumin | KQFTKAELSQ LLKDIDGYGG IALPELIATM FHTSGYDTQ |
| | Alpha1 39mer - Cys | |
| | to Ala | |
| 7 | Alpha-lactalbumin | LDDDITDDIM XAKKILDIKG IDYWLAHKAL |
| | Alpha2 - modified | XTEKLEQWLX EKL |
| | Cys | |
| 8 | Alpha-lactalbumin | LDDDITDDIM AAKKILDIKG IDYWLAHKAL |
| | Alpha2 - Cys to Ala | ATEKLEQWLA EKL |
| 9 | Sar1 23mer | MAGWDIFGWF RDVLASLGLW NKH |
| 10 | Alpha-lactalbumin | KQFTKAELSQ LLKDI |
| | 15mer | |
| 11 | Sar1 15mer | MAGWDIFGWF RDVLA |

In a particular embodiment, the polypeptide is a recombinant protein having the sequence of native mature alpha-lactalbumin but which has all of the cysteines found at positions 6, 28, 61, 73, 77, 91, 111 and 120 in the full length sequence of mature human alpha-lactalbumin mutated to other amino acids, such as alanine, which do not give rise to disulphide bridges. Thus a particular of a protein that may be utilised in accordance with the invention comprises a protein of SEQ ID NO 1 or 2.

As reported in WO2010079362, additional amino acid residues, for example up to 20 amino acids, may be attached at N and/or C terminal of the protein, if convenient, for example for expression purposes. Thus in particular, a recombinant protein as shown in SEQ ID NO. 1 or 2 but with an additional methionine at the N-terminus (SEQ ID NO 3 or 4) can be used in accordance with the invention.

In a particular embodiment, the complex comprises amino acids of the Alpha 1 (residues 1-39) or Alpha 2 (residues 81-123) of human alpha-lactalbumin wherein the cysteines are replaced with other amino acids such as alanine, to prevent any intra-molecular bonding. Thus the peptide may be of SEQ ID NO 5 or SEQ ID NO 7 where X is an amino acid residue other than cysteine. A particular example of such sequences are those of SEQ ID NO 6 or SEQ ID NO 8. In a particularly preferred embodiment, the polypeptide sequence comprises or consists of SEQ ID NO 6.

It is known that complexes obtained using α-lactalbumin from sources other than human milk, and in particular, BAMLET, obtained using bovine α-lactalbumin shows qualitatively similar effects on cells and in particular on tumour cells as HAMLET (see for instance, Rammer et al. (2010) Mol. Cancer Ther. 9(1) 24-32).

Other peptides may also be used in the complex and the suitability may be tested by determining whether complexes with a fatty acid salt are active, for instance in killing cells using methods as described hereinafter.

In another embodiment, the peptide is derived from the COPII family protein SAR1. A particular example of such a peptide is a peptide of SEQ ID NO 9. As reported in WO 2019/243547, biologically active complexes have been formed with fragments that are around 15 amino acids in length. Particular examples of such polypeptides are polypeptides of SEQ ID NOs 10 and 11.

In another embodiment, the polypeptide element is a naturally-occurring protein or a synthetic form thereof, in particular an alpha-lactalbumin, such as human, bovine, sheep, camel or goat alpha-lactalbumin. In a preferred embodiment, the protein is human alpha-lactalbumin or bovine alpha-lactalbumin.

Other peptides may also be used in the complex and the suitability may be tested by determining whether complexes with a fatty acid salt are active, for instance in opening potassium ion channels and/or killing cells using methods as described hereinafter.

As used herein, the term “biologically active” means that the complex has a biological activity, which is different from, or stronger than, the individual components. In particular, the complex is able to induce cell death in particular selectively in tumour cells and/or has a bactericidal or antiviral effect not seen with the native protein including for example monomeric α-lactalbumin forms, although other therapeutic effects may be available.

Suitably fatty acids or lipids or pharmaceutically acceptable salts include those known to provide biologically active complexes. These include fatty acids or lipids for example as described in WO2008058547. Where salts are used, these are suitably water soluble salt. Particular examples of suitable salts may include alkali or alkaline earth metal salts. In a particular embodiment, the salt is an alkali metal salt such as a sodium- or potassium salt. Where used in pharmaceuticals, the salts will be pharmaceutically acceptable, and will be suitable for food use when used in nutraceuticals.

In a preferred embodiment, a fatty acid or a pharmaceutically acceptable salt thereof is used with the invention. Particular examples of fatty acids or fatty acid salts used in the present invention are those having from 4-30, for example from 6 to 28, such as from 8 to 26 carbon atoms. In particular embodiments, the fatty acid has from 10 to 24, such as from 12 to 22, for example from 14 to 20 carbon atoms. In particular, the fatty acid will have 16, 17, 18 or 20 carbon atoms. The fatty acids may be saturated or unsaturated. Preferably the fatty acids are unsaturated and may be polyunsaturated. In a preferred embodiment, the fatty acid is monounsaturated.

In particular however, the complexes of the invention utilize fatty acids or salts of fatty acids having 18 carbon atoms. A specific example is oleic acid or oleate salt. In particular oleic acid or oleate used in the method of the invention is C18:1 oleic acid of formula CH₃(CH₂)₇CH═CH(CH₂)₇COOH or CH₃(CH₂)₇CH═CH(CH₂)₇COO⁻. In one embodiment, oleic acid is used in the methods and compositions of the invention.

In a particular embodiment, a pharmaceutically acceptable salt of oleic acid is used in the process. Suitable pharmaceutically acceptable salts would be understood in the art.

Use of a salt, and in particular a water-soluble salt of the oleic acid, fatty acid or lipid means that the preparation of the biologically active complex may be further facilitated since aqueous solutions may be formed more readily. Suitable water-soluble salts are alkali or alkaline-earth metal salts such as sodium or potassium salts.

Furthermore, it has been found that salts and in particular oleate salts such as sodium oleate appear to have some inherent tumoricidal effect. Therefore the inclusion of this in the complex may give rise to activity increases.

In a particular embodiment, the first salt used in the buffer component of the invention is sodium chloride.

In another particular embodiment, the second salt used in the buffer component of the invention is disodium phosphate.

In another particular embodiment, the third salt used in the buffer component of the invention is mono-potassium phosphate.

The ratio of first salt:second salt used in the method of the invention is suitably from 8:1 to 1:1, for example from 5:1 to 2:1 and in particular from 4:1 to 3.5:1. Where present the ratio of first salt:third salt is from 20:1 to 5:1, for example from 15:1 to 10:1 such as from 12.5:1 to 11.5:1.

In a particular embodiment the ratio of first to second to third salt is 13-12:4-3:1.

The ratio of oleic acid or oleate:peptide mixed in the method of the invention is suitably in the range of from 20:1 to 1 to 1, but preferably an excess of oleate is present, for instance in a ratio of oleate:peptide of about 5:1. The mixing can be carried out at a temperature of from 0-50° C., conveniently at ambient temperature and pressure.

Preferred amounts of the components are provided below. The amounts are given in terms of concentration, which refer to the intended concentration after the dry composition is made up with liquid. The fatty acid or lipid, or pharmaceutically acceptable salt thereof, can be added in an amount of at least 0.01 mM, 0.1 mM or 1 mM and/or at most 100 mM, 50 mM or 25 mM. The polypeptide can be added in an amount of at least 0.01 mM, 0.1 mM or 1 mM and/or at most 100 mM, 50 mM or 25 mM. Each salt component can be added in an amount of at least about 0.1, 0.5, 1, or 2 g/L and at most about 50, 40, 30, 20, or 10 g/L. In one embodiment, the total concentration of the salts in the buffer component of the composition is at least about 0.5, 1, 2, 5 or 10 g/L and at most about 150, 120, 100, 80, 60 or 40 g/L. Typically, no more than about 10, 9, 8, 7, 6, 5, 4 or 3 salts will be added as the buffer component of the composition.

The invention also relates to a method for preparing a biologically active complex from the composition of the invention by adding water and agitating the mixture. Agitation can be performed at a moderate temperature.

As used herein, the expression ‘moderate temperature’ refers to temperatures of up to 50° C., for example from 0-50° C., for example from 10-40° C., and more particularly from 15-25° C., such as at ambient temperature. Although the mixture may be warmed for example to temperatures of up to 50° C., such as up to 40° C. to achieve rapid dissolution, there is no need to heat the solution extensively such as described in by boiling, provided only that a suitable buffer component is present in the composition. Thus in a particular embodiment, the method is carried out at ambient temperature. As such, the method can typically be conducted at room temperatures as found in residential or medical environments, obviating the need for heating equipment. Such temperatures are generally below the ‘melt temperature’ at which the polypeptides become unfolded or denatured. However, the applicants have found that they are still able to form biologically active complexes using the compositions and methods of the invention.

Dissolution is facilitated by agitation, for example by hand shaking or vortexing. If required, the solution may be filtered through a sterile filter at this stage. Suitable filters include polyethersulfone membranes (PES) or Minisart® NML Cellulose acetate membranes.

Any such agitation processes will be carried out for a period of time sufficient to ensure the dissolution of the elements in the buffer component. Although the precise timings may vary depending upon factors such as the particular nature of the polypeptide being used and the temperature at which the mixture is held, the timings will typically be quite short, for example no more than 10 minutes, for example from 1-5 minutes such as about 2 minutes.

The compositions of the invention may be formulated into useful pharmaceutical compositions by combining them with pharmaceutically acceptable carriers in the conventional manner. Typically, the compositions would be combined with water or an aqueous liquid solution or suspension. Such pharmaceutical compositions form a further aspect of the invention.

The pharmaceutical compositions in accordance with the invention are suitably pharmaceutical compositions in a form suitable for topical use, for example as creams, ointments, gels, or aqueous or oily solutions or suspensions. These may include the commonly known carriers, fillers and/or expedients, which are pharmaceutically acceptable.

Topical solutions or creams suitably contain an emulsifying agent for the protein complex together with a diluent or cream base.

The daily dose of the complex varies and is dependent on the patient, the nature of the condition being treated etc. in accordance with normal clinical practice. As a general rule from 2 to 200 mg/dose of the biologically active complex is used for each administration.

In a further aspect of the invention, there is provided a method for treating cancer which comprises administering to a patient in need thereof, a biologically active complex as described above.

In particular, the complex may be used to treat cancers such as human skin papillomas, human bladder cancer and glioblastomas. In the latter case, administration may be by infusion as is known in the art.

The invention further provides the biologically active complex as defined above for use in therapy, in particular in the treatment of cancer.

The complex may also be of use in the prevention of cancer, in particular gastrointestinal cancer as described for example in WO2014/023976. In this case, the complex may be combined with a foodstuff, such as a dairy product such as yoghurt for use as a nutraceutical. Compositions of this type form a further aspect of the invention.

The composition of the invention is particularly suited to use with dry or powdered nutraceutical compositions. Dry or powdered nutraceutical compositions can be supplemented with the composition of the invention, to give a nutraceutical composition comprising the composition of the invention. As such, when the nutraceutical composition is prepared as a substance for consumption by the addition of a suitable liquid (such as water), the biologically active complex also forms within the nutraceutical composition. The invention therefore provides for a very convenient way to supplement a dry or powdered nutraceutical composition with the ability to form a biologically active complex according to the invention.

Where a food product or nutraceutical composition is sold in the liquid or gel form (i.e. a dairy product such as milk or yoghurt, or as a health drink) the composition of the invention can be readily introduced to the food product or nutraceutical composition during manufacture. The composition of the invention can be introduced directly to the food product or nutraceutical composition, or the composition of the invention can first be mixed with a suitable liquid (e.g. water) and subsequently added to the food product or nutraceutical composition.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

The invention will now be particularly described by way of example with reference to the accompanying drawings in which

FIG. 1 shows a schematic of a composition according to the invention and a method of preparing a biologically active complex by adding sterile water and shaking.

FIG. 2 shows the results of ATP Lite and PrestoBlue studies obtained using a biologically active complex prepared according to the schematic of FIG. 1 and a control biologically active complex on tumour cells as described below.

### EXAMPLE 1

Production of Biologically Acceptable Complexes

A composition according to the invention was prepared using a peptide of SEQ ID NO 6, which is a variant of a fragment of human α-lactalbumin.

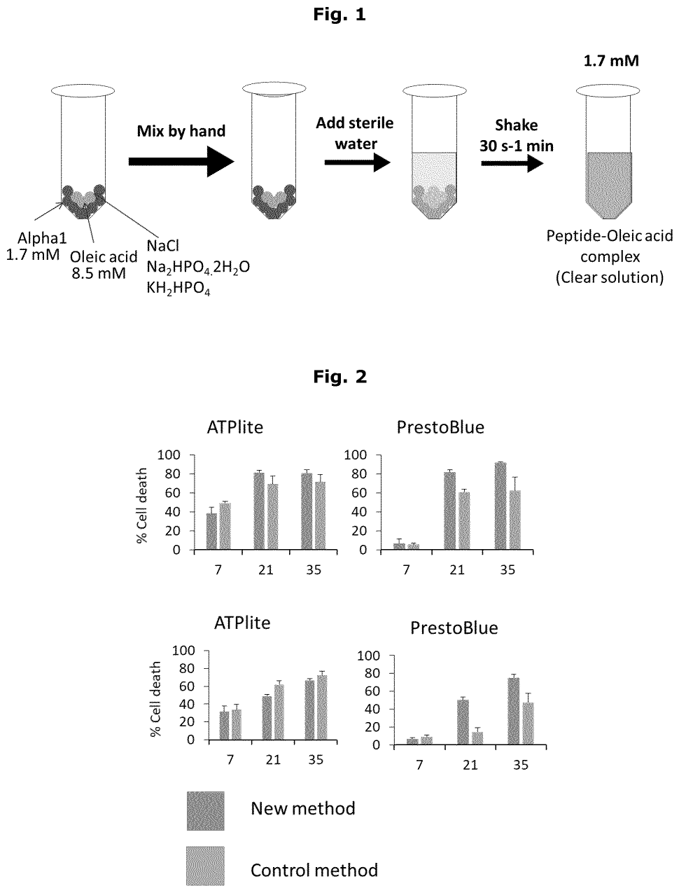

As shown in FIG. 1, the peptide (1.7 mM), in lyophilised form, was added to a tube together with pure oleic acid (8.5 mM) and NaCl (6.8 g/L), Na₂HPO₄×2H₂O (4.8 g/L); and KH₂PO₄ (1.3 g/L). The concentrations provided are with respect to the concentration once made up with an appropriate quantity of water. The tube was then mixed by hand.

To prepare the biologically active complex, sterile water was added to the tube and shaken by hand for 30 to 60 seconds. The result was a clear solution, indicating that the biologically active complex had been formed without any precipitation of protein.

### EXAMPLE 2

Cell Death Assay

Human lung carcinoma cells (A549, ATCC) were cultured in RPMI-1640 with non-essential amino acids (1:100), 1 mM sodium pyruvate, 50 μg/ml Gentamicin and 5-10% fetal calf serum (FCS) at 37° C., 5% CO₂. For cell death experiment, cells were grown on 96-well plate (2×10⁴/well, Tecan Group Ltd) overnight. Cells were incubated with biologically active complexes obtained in Example 1 at dosages equivalent to either 7, 21 or 35 μM peptide in serum-free RPMI-1640 at 37° C. FCS was added after 1 hour. Cell death was quantified 3 hours after peptide-oleate treatment by two biochemical methods including 1) estimation of cellular ATP levels using luminescence based ATPlite™ kit (Perkin Elmer) and 2) Presto Blue fluorescence staining (Invitrogen, A13262). Fluorescence and luminescence was measured using a microplate reader (Infinite F200, Tecan).

The results are shown in FIG. 2. FIG. 2 shows a comparison of tumour cell death activity of biologically active complex prepared according to the invention with biologically active complex prepared according to the technique of WO 2018/210759. The results show comparable activity, indicating that the composition of the invention is fully capable of being used to form high levels of biologically active complex.

1. A method for preparing a composition, the method comprising the steps of combining:
a. a dried polypeptide,
b. a fatty acid or lipid, or a pharmaceutically acceptable salt thereof, and
c. a buffer component comprising at least two salts, preferably wherein the first salt is sodium or potassium chloride and the second salt is disodium phosphate or mono-potassium phosphate,
wherein the composition is substantially water-free.

2. A method according to claim 1 wherein the buffer component further comprises a third salt which is mono-sodium or mono-potassium phosphate, preferably mono-potassium phosphate.

3. A method according to any preceding claim, wherein the polypeptide is a membrane-perturbing protein.

4. A method according to any preceding claim, wherein the polypeptide is selected from natural alpha-lactalbumin, SAR1, or lysozyme, or active fragments or active variants thereof.

5. A method according to claim 4, wherein the alpha-lactalbumin is human alpha-lactalbumin or bovine alpha-lactalbumin.

6. A method according to any preceding claim, wherein the active fragment or active variant comprises a polypeptide which lacks intra-molecular bonds.

7. A method according to any preceding claim, wherein the active fragment or active variant is a fragment or variant of up to 50 amino acids.

8. A method according to any preceding claim, wherein the active fragment comprises a polypeptide of any of SEQ ID NOs 5 to 11, preferably a polypeptide of SEQ ID NO 6.

9. A method according to any preceding claim, wherein the first salt is sodium chloride and the second salt is disodium monophosphate.

10. A method according to any preceding claim, wherein the fatty acid is an unsaturated fatty acid, preferably a C18 unsaturated fatty acid, more preferably a cis C18:1:9 or C18:1:11 fatty acid, yet more preferably oleic acid, or a pharmaceutically acceptable salt thereof.

11. A composition obtainable by a method according to any one of the preceding claims.

12. A composition that is substantially water-free comprising:
a. a dried polypeptide,
b. a fatty acid or a lipid, or a pharmaceutically acceptable salt thereof, and
c. a buffer component comprising at least two salts, preferably wherein the first of which is sodium or potassium chloride and the second of which is disodium phosphate or mono-potassium phosphate,
wherein the composition is substantially free of polypeptide that is in a complex with the fatty acid or a pharmaceutically acceptable salt thereof.

13. A composition according to claim 12, the composition further comprising the features of any of claims 2 to 10.

14. A method for preparing a biologically active complex, the method comprising the steps of providing a composition according to any of claims 11 to 13, adding an aqueous carrier, and agitating the mixture.

15. A method according to claim 14 wherein the agitation is carried out at a moderate temperature, preferably from 10-40° C., more preferably at ambient temperature.

16. A biologically active complex obtainable by any of claims 14 to 15.

17. A biologically active complex obtainable by any of claims 14 to 15, or the composition of any of claims 11 to 13, for use in therapy.

18. A biologically active complex or composition for use according to claim 17, wherein the use is treatment or prevention of cancer or viral infection.

19. A pharmaceutical or nutraceutical composition comprising a composition according to claims 11 to 13 or a biologically active complex of claim 16.

20. A method for treating or preventing cancer or a viral infection, said method comprising administering to a patient in need thereof an effective amount of a biologically active complex according to claim 16 or a composition according to any of claims 11 to 13.
